(19) United States
(12) Patent Application Publication
(10) Pub. No.: US 2007/0038267 A1
(43) Pub. Date: Feb. 15, 2007

(54) Vision regeneration assisting apparatus

(75) Inventors: Kenzo Shodo, Kyoto-shi (JP); Akihiro Uehara, Nara-shi (JP); Jun Ohta, Souraku-gun (JP); Takashi Tokuda, Kyoto (JP)

Correspondence Address: SUGHRUE MION, PLLC, 2100 PENNSYLVANIA AVENUE, N.W., SUITE 800, WASHINGTON, DC, 20037, US

(73) Assignee: NIDEK CO., LTD.

(21) Appl. No.: 11/502,413

(22) Filed: Aug. 11, 2006

(30) Foreign Application Priority Data
Aug. 11, 2005 (JP) P2005-232878

(51) Int. Cl. A61N 1/32

(52) U.S. Cl. 607/54

(57) ABSTRACT

A vision regeneration assisting apparatus includes: a substrate which is to be placed in a body of a patient; a plurality of electrode groups each having a plurality of electrodes which are arranged on the substrate, the electrodes applying electrical stimulation pulse signals to cells forming a retina of the patient; and a plurality of switch units which are arranged on the substrate and of which one is provided for each electrode group, the switch units selectively switching output of the electrical stimulation pulse signals from each of the electrodes.

### BACKGROUND OF THE INVENTION

The present invention relates to a vision regeneration assisting apparatus for assisting regeneration (restoration) of vision.

Recent years, a vision regeneration assisting apparatus for recovering lost vision of a patient who has lost his/her vision by installing (placing) an electrode in his/her body (eye) to give (apply) electrical stimulation (electrical stimulation pulse signal) to cells that forms a retina is proposed. In such an apparatus, it is preferable that a number of electrodes can be installed in the body in order to make the patient obtain the vision of high resolution.

However, the more the number of electrodes is, the larger a unit to be installed in the body and connected to the electrodes must be, which increases a burden not only to the patient as a matter of course, but also to an operator.

### SUMMARY OF THE INVENTION

It is a technical object of the invention to provide a vision regeneration assisting apparatus in which increase in size of a unit to be installed in a body caused by the installation of a number of electrodes can be suppressed.

In order to solve the above-described problem, the present invention is characterized by the following configuration.
(1) A vision regeneration assisting apparatus comprising:

a substrate which is to be placed in a body of a patient;

a plurality of electrode groups each having a plurality of electrodes which are arranged on the substrate, the electrodes applying electrical stimulation pulse signals to cells forming a retina of the patient; and

a plurality of switch units which are arranged on the substrate and of which one is provided for each electrode group, the switch units selectively switching output of the electrical stimulation pulse signals from each of the electrodes.
(2) The vision regeneration assisting apparatus according to (1), wherein the switch units are arranged on a side of the substrate opposed to a side where the electrodes are arranged.
(3) The vision regeneration assisting apparatus according to (1), wherein the switch unit comprises a multiplexer.
(4) The vision regeneration assisting apparatus according to (1), wherein the vision regeneration assisting apparatus is of an external photographing type, and further comprises a photographing unit for photographing an external object to be recognized by the patient.
(5) The vision regeneration assisting apparatus according to (4) further comprising a control unit which is to be placed in the body and generates the electrical stimulation pulse signals based on image data obtained by the photographing unit or data obtained by subjecting the image data to a predetermined process, and transmits the generated electrical stimulation pulse signals to each of the switching units.
(6) The vision regeneration assisting apparatus according to (5), wherein the switching units and the control unit are arranged on a side of the substrate opposed to a side where the electrodes are arranged.

### BRIEF DESCRIPTION OF THE DRAWINGS

FIG. 1 is a schematic diagram showing an external device of a vision regeneration assisting apparatus according to an embodiment of the invention.

FIGS. 2A and 2B illustrate schematic diagrams showing an internal device of the vision regeneration assisting apparatus.

FIGS. 3A and 3B illustrate enlarged drawings of the internal device.

FIG. 4 is a drawing showing a state in which the internal device is installed in the body (in an eye) of a patient.

FIG. 5 is a schematic block diagram of a control system of the vision regeneration assisting apparatus.

### DETAILED DESCRIPTION OF PREFERRED EMBODIMENTS

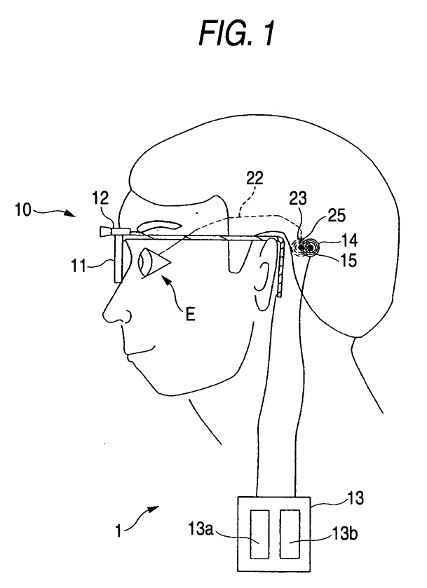

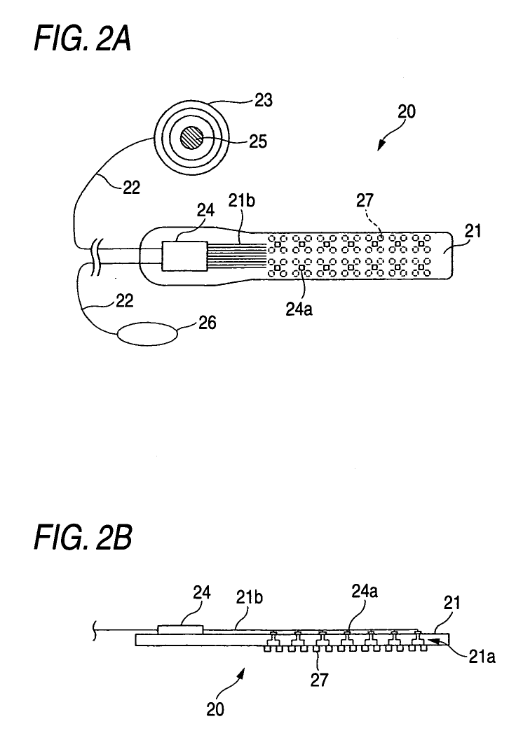

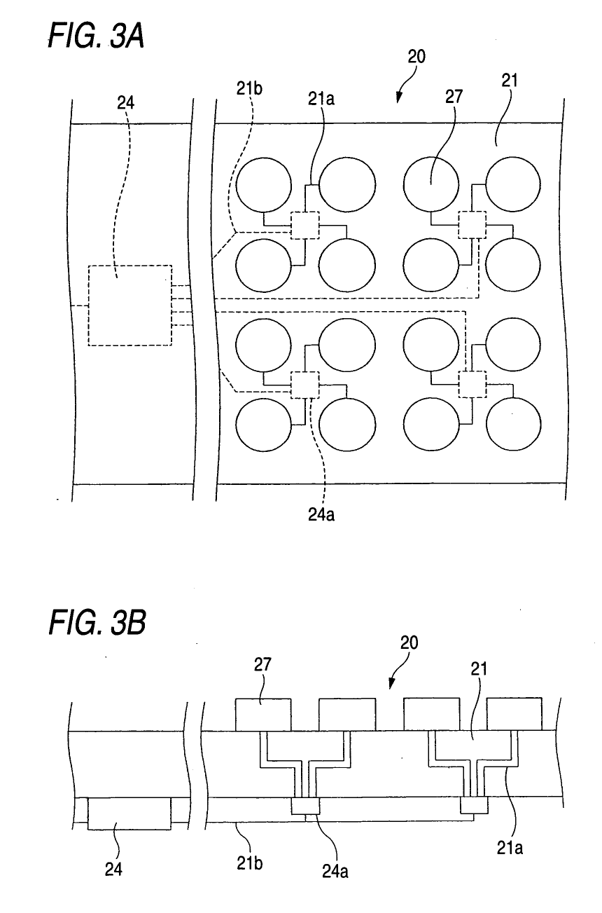

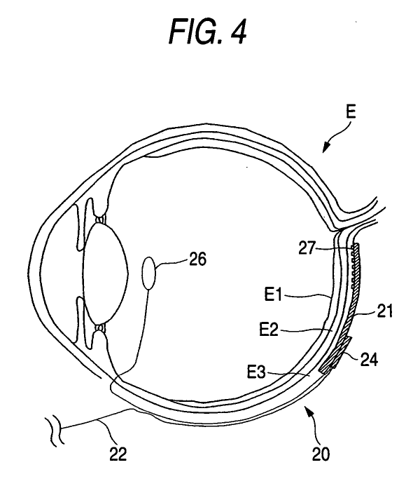

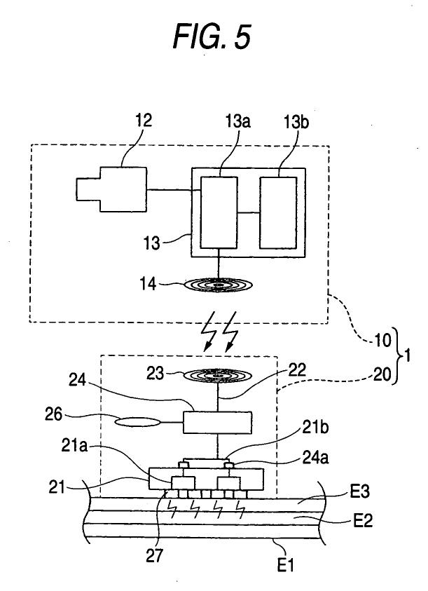

Referring now to the drawings, an embodiment of the invention will be described. FIG. 1 is a schematic diagram of an external device of a vision regeneration assisting apparatus according to an embodiment of the invention. FIGS. 2A and 2B are schematic diagrams showing an internal device of the vision regeneration assisting apparatus. FIG. 2A is a plan view of the internal device viewed form the opposite side from the electrode-arranged side, and FIG. 2B is a cross-sectional view with the electrode-arranged side faced downward and viewed from the side. FIGS. 3A and 3B are enlarged drawings of the internal device. FIG. 3A is a plan view of the internal device viewed from the electrode-arranged side, and FIG. 3B is a cross-sectional view with the electrode-arranged side faced upward and viewed from the side. FIG. 4 is a drawing showing a state in which the internal device is installed in a body (in an eye) of a patient. FIG. 5 is a schematic block diagram of a control system of the vision regeneration assisting apparatus.

A vision regeneration assisting apparatus 1 includes an external device 10 for photographing an external object, an internal device 20 for applying electrical stimulation to cells forming a retina E1 to assist regeneration (restoration) of the vision. The external device 10 includes a visor 11 to be worn by the patient, a photographing unit 12 including a CCD camera or the like mounted to the visor 11, an external unit 13, and a transmitting unit 14 including a primary coil. The visor 11 has a shape of eyeglasses, and is used by being worn in front of the patient's eye E. The photographing unit 12 is mounted to the front surface of the visor 11 and photographs an object to be recognized by the patient.

The external unit 13 includes a processing unit 13a having an arithmetic processing circuit, and a power source unit (battery) 13b for supplying electric power for the apparatus 1 (external device 10 and internal device 20). The processing unit 13a processes image data obtained by the photographing unit 12, and converts into electrical stimulation pulse signal data (data such as the strength of electrical stimulation pulse signal or the position of stimulation). The transmitting unit 14 transmits the electrical stimulation pulse signal data converted by the processing unit 13a and the electric power (electric power data) from the power source unit 13b via the processing unit 13a to the internal device 20 as an electromagnetic wave. A magnet 15 is mounted to the center of the transmitting unit 14. The magnet 15 improves the data transmission efficiency by the transmitting unit 14 and is also used for fixing the position with respect to a receiving unit 23 described later.

The internal device 20 includes a substrate 21 on which a plurality of electrodes 27 for applying the electrical stimulation to the cells forming the retina E1 are arranged (arrayed), a cable 22, the receiving unit 23 including a secondary coil, an internal unit 24 as a control unit arranged on the substrate 21, and a plurality of multiplexers 24a as switch units arranged on the substrate 21, and an indifferent electrode 26. The substrate 21 is formed of a material which is good in biocompatibility such as polypropylene or polyimide, and is capable of being bent at a predetermined thickness into a thin and long plate shape.

The plurality of electrodes 27 are arranged on the substrate 21 in a substantially equidistance grid-like pattern, and form an electrode array. The electrodes 27 are formed of conductive material which is biocompatible and is superior in anti-corrosion property, for example, metal such as gold or white gold.

The receiving unit 23 receives the electrical stimulation pulse signal data and the electric power data transmitted from the external device 10. A magnet 25 is mounted to the center of the receiving unit 23. Accordingly, the transmitting unit 14 on the skin is magnetically attracted by the receiving unit 23 embedded (placed) under the skin of the patient's temporal region, and hence the transmitting unit 14 is held on the temporal region. In this embodiment, although the transmitting unit 14 and the receiving unit 23 are set to the patient's temporal region, the invention is not limited thereto. What is essential is being capable of transmitting the electrical stimulation pulse signal data and the electric power data from outside the body to the inside of the body. For example, it is also applicable to mount the transmitting unit 14 to the visor 11 in front of the eye E, and the receiving unit 23 in the eye E so as to oppose the transmitting unit 14 (for example, near the anterior portion of the eye E).

The internal unit 24 includes a circuit for separating the electrical stimulation pulse signal data and the electric power data which are received by the receiving unit 23 and transmitted via the cable 22, a circuit for converting the electrical stimulation pulse signal data into the electrical stimulation pulse signal and the position signal thereof, a circuit for sending the electrical stimulation pulse signal and the position signal thereof to the each multiplexer 24a, and the like. The internal unit 24 obtains drive power for the internal device 20 by the received electric power data transmitted via the cable 22.

An electrode group is constituted by four adjacent electrodes 27 as shown in FIG. 3A, and the four electrodes 27 are connected to one multiplexer 24a via lead wires 21a independently. The multiplexer 24a of each electrode group is connected to the internal unit 24 independently via a lead wire 21b. The multiplexers 24a are arranged on the side of the substrate 21 opposite from the side where the electrodes 27 are arranged, and the internal unit 24 is arranged on the side of the substrate 21 on which the multiplexers 24a are arranged.

Each of the multiplexers 24a receives the electrical stimulation pulse signal and the position signal thereof sent from the internal unit 24, obtains information on the position of stimulation based on the position signal, and selects the electrode 27 for outputting the electrical stimulation pulse signal based on the position information. Then, the multiplexer 24a brings only the selected electrode 27 into a conductive state by the switching element thereof and outputs the electrical stimulation pulse signal therefrom.

In this manner, a predetermined number (plurality) of adjacent electrodes 27 are grouped as one electrode group and are connected to one multiplexer 24a, and the plurality of multiplexers 24a are dispersed on the substrate 21. Accordingly, even when the number of the electrodes 27 increases, a multi-channeled and highly-integrated unit is achieved without providing large scaled wiring by copying such a configuration. For example, when the number of electrodes is 10000 arranged in rows and columns of 100×100, 2500 pieces of multiplexers 24a are provided and the outputs of the electrical stimulation pulse signal from 10000 pieces of electrodes 27 are selectively switched by 2500 pieces of the multiplexers 24a.

As described above, with the configuration in which the predetermined number of the adjacent electrodes are grouped as one electrode group and connected to one multiplexer, the function of the multiplexer can be divided, and hence the multiplexers can be downsized. With the configuration in which the multiplexers are arranged on the side of the substrate opposite from the side where the electrodes are arranged, the multiplexers can be downsized without depending on the size of the electrodes. Further, the electrodes can be arranged on the substrate without depending on the size and the positions of the multiplexers. Since the multiplexers can be downsized, it is not necessary to increase the thickness of the multiplexers for providing bending strength sufficient for resisting bending along an eyeball when being embedded in the body (in the eye). Therefore, the thickness of the multiplexers can be reduced, and the thickness of the entire internal device can be reduced.

Incidentally, the number of the electrodes 27 in one electrode group is not limited to four, and the number of the electrodes 27 in the respective electrode groups is not limited to be the same. Any number of the electrodes 27 is applicable as long as the function of the multiplexer can be divided and the multiplexers 24a can be downsized. In this case, the size (thickness) of the multiplexers 24a may be set to an extent that is not inconvenient for embedding in the body. It is also applicable that the multiplexers 24a are connected to each other, and some of the multiplexers 24a are connected to the internal unit 24 instead of the configuration in which the respective multiplexers 24a are connected to the internal unit 24 independently.

The distance between the multiplexer 24a and the electrodes 27 connected thereto is not limited to the equal distance, and any distance is applicable as long as they are sufficiently close for easy wiring.

The arrangement pattern (layout) of the electrodes 27 is not limited to the substantially equidistance grid-like pattern, and may be any pattern as long as it can desirably stimulate the cells forming the retina E1.

The internal unit 24 and the multiplexers 24a are coated with a material which is biocompatible and superior in anti-corrosion property, for example, metal such as gold or white gold so as to ensure high air-tightness.

Other components of the internal device 20 except for the electrodes 27 and the indifferent electrode 26 are coated with a coating agent which is biocompatible such as silicon, parylene, polyimide and so on.

The internal device 20 in this embodiment is embedded (placed) outside a screla E3 so as to come into abutment with the screla E3 as shown in FIG. 4. However, since the internal unit 24 and the multiplexers 24a are arranged on the side of the substrate 21 opposite from the side on which the electrodes 27 are arranged, the internal unit 24 and the multiplexers 24a do not abut with the screla E3 when embedded. Therefore, the internal device 20 can be embedded easily.

The embedding position of the internal device 20 is not limited to the outside the screla E3, and may be outside a choroid E2 (between the choroid E2 and the screla E3), outside the retina E1 (between the retina E1 and the choroid E2), inside the retina E1 (on the retina E1), and so on as long as the electrodes 27 are arranged at positions where the cells forming the retina E1 can be preferably stimulated. For example, the portion of the substrate 21 where the electrodes 27 are arranged is positioned outside the choroid E2 and the portion of the substrate 21 where the internal unit 24 is arranged is positioned outside the screla E3.

The cable 22 coated with a material having insulating property and good biocompatibility is led under the skin along the temporal region from the receiving unit 23 toward the eye E and passed through inside the upper eyelid of the patient and inserted into the eye pit as shown in FIG. 1 and FIG. 4. The cable 22 inserted into the eye pit is passed through outside the screla E3 (or inside) and is connected to the internal unit 24 arranged on the substrate 21 as shown in FIG. 4.

The cable 22 is then led from the internal unit 24, passed through a ciliary ring and is embedded in the eye E (within a vitreous body) in such a manner that the distal end of the cable 22 opposes the electrodes 27 arranged on the substrate 21 with the intermediary of the retina E1 and the like. The ring-shaped indifferent electrode 26 is connected to the distal end of the cable 22. The shape of the indifferent electrode 26 is not limited to the ring shape. Although the indifferent electrode 26 is embedded in the eye E in this embodiment, the invention is not limited thereto, and any position is applicable as long as the electrical stimulation pulse signal output from the electrodes 27 can be provided to the cells forming the retina E1 efficiently. The indifferent electrode 26 is formed of a conductive material which is biocompatible and superior in anti-corrosion property, for example, metal such as gold or white gold.

Next, manufacture of the internal device 20 will be described. The internal unit 24 and the plurality of multiplexers 24a are made based on semiconductor IC technology, and terminals are exposed from a portion thereof to be jointed with the substrate 21. The lead wires 21a are wired inside the substrate 21 by RIE (reactive ion etching). The lead wires 21b are wired by laminating (vapor deposing) a conductive material, which is biocompatible and superior in anti-corrosion property, for example, metal such as gold or white gold, on an outside surface of the substrate 21. Here, one side surface of the substrate 21 is referred as a surface on which the electrodes 27 are arranged (formed), and the lead wires 21a are exposed from the arranging portions thereof. The other side surface of the substrate 21 is referred as a surface where the internal unit 24 and the multiplexers 24a are arranged, and the lead wires 21a are exposed from the arranging portions thereof. The terminals of the internal unit 24 and the respective multiplexers 24a are joined (flip chip joining) to the lead wires 21a exposed from the substrate 21. Further, the internal unit 24 is connected to each of the multiplexers 24a through the lead wires 21b. The electrodes 27 are formed, for example, by sputtering, on the lead wires 21a exposed from the arranging surface of the substrate 21 where the electrodes 27 are formed. The internal unit 24 is connected to the receiving unit 23 by the cable 22.

Subsequently, an operation of the apparatus 1 configured as described above will be described.

The image data obtained by the photographing unit 12 is entered into the processing unit 13a, is converted into signals within a predetermined frequency band (electrical stimulation pulse signal data) by the processing unit 13a, and is transmitted to the internal device 20 by the transmitting unit 14.

The electric power supplied from the power unit 13b is converted into signals (electric power data) within a predetermined frequency band different from the electrical stimulation pulse signal data by the processing unit 13a, and is transmitted to the internal device 20 together with the electrical stimulation pulse signal data by the transmitting unit 14.

The electrical stimulation pulse signal data and the electric power data transmitted from the external device 10 are received by the receiving unit 23, are entered into the internal unit 24, are divided into the electrical stimulation pulse signal data and the electric power data by the internal unit 24, are converted into the electrical stimulation pulse signal and the position signal thereof based on the electrical stimulation pulse signal data, and are transmitted to the respective multiplexers 24a.

The respective multiplexers 24a selectively switch the outputs from the electrical stimulation pulse signals from the connected respective electrodes 27 based on the transmitted position signal. The electrical stimulation pulse signals output from the respective electrodes 27 stimulates the cells forming the retina E1, such as bipolar cells and ganglion cell retina. Accordingly, the patient can recognize the object photographed by the photographing unit 12.

In the embodiments shown above, the vision regeneration assisting apparatus of an external (extra-ocular) photographing type in which the photographing unit is provided outside the body (outside the eye) has been described. However, the invention is not limited thereto, and the invention may be applied also to the vision regeneration apparatus of an internal (intra-ocular) photographing type in which an optical sensor such as a photodiode is embedded in the body (in the eye).

What is claimed is:

1. A vision regeneration assisting apparatus comprising:
a substrate which is to be placed in a body of a patient;
a plurality of electrode groups each having a plurality of electrodes which are arranged on the substrate, the electrodes applying electrical stimulation pulse signals to cells forming a retina of the patient; and
a plurality of switch units which are arranged on the substrate and of which one is provided for each electrode group, the switch units selectively switching output of the electrical stimulation pulse signals from each of the electrodes.

2. The vision regeneration assisting apparatus according to claim 1, wherein the switch units are arranged on a side of the substrate opposed to a side where the electrodes are arranged.

3. The vision regeneration assisting apparatus according to claim 1, wherein the switch unit comprises a multiplexer.

4. The vision regeneration assisting apparatus according to claim 1, wherein the vision regeneration assisting apparatus is of an external photographing type, and further comprises a photographing unit for photographing an external object to be recognized by the patient.

5. The vision regeneration assisting apparatus according to claim 4 further comprising a control unit which is to be placed in the body and generates the electrical stimulation pulse signals based on image data obtained by the photographing unit or data obtained by subjecting the image data to a predetermined process, and transmits the generated electrical stimulation pulse signals to each of the switching units.

6. The vision regeneration assisting apparatus according to claim 5, wherein the switching units and the control unit are arranged on a side of the substrate opposed to a side where the electrodes are arranged.
